(19) United States
(12) Patent Application Publication
(10) Pub. No.: US 2004/0018601 A1
(43) Pub. Date: Jan. 29, 2004

(54) Method for generating pure populations of mobile mebrane-associated biomolecules on supported lipid bilayers

(76) Inventors: Steven G. Boxer, Stanford, CA (US); Lance Kam, Palo Alto, CA (US)

Correspondence Address: BOZICEVIC, FIELD & FRANCIS LLP, 200 MIDDLEFIELD RD, SUITE 200, MENLO PARK, CA, 94025, US

(21) Appl. No.: 10/370,423

(22) Filed: Feb. 21, 2003

Related U.S. Application Data
(60) Provisional application No. 60/358,941, filed on Feb. 21, 2002.

(51) Int. Cl. C12N 9/00; C12N 1/00; C07K 14/705

(52) U.S. Cl. 435/183; 435/317.1; 530/350

(57) ABSTRACT

Methods are provided for generating mobile, membrane-associated biomolecules in a supported membrane, by the process of sequestering and collecting mobile and immobile populations of biomolecules. Populations of mobile biomolecules, but not those that are immobile, can be moved to a region through a variety of mechanisms, including passive diffusion or induced drift, generating a pure, mobile population of biomolecules. In some embodiments of the invention, complex substrates are utilized, e.g. substrates containing switchable barriers to lipid diffusion, including fluidic channels; removal of regions of lipid bilayer by blotting, and the like.

### INTRODUCTION

Membrane-associated biomolecules mediate a wide variety of cellular functions. It is believed that as much as ⅔ of the human genome encodes for proteins that either span or are anchored to these membranes. Consequently, the ability to examine membrane-associated biomolecules and the binding of ligands to membrane receptors in an easily manipulated system is of great and increasing interest. It is critical that these systems capture the lateral fluidity of biological membranes as many chemical signals such as hormones bind to multiple receptors and many cell functions are mediated by multivalent molecular interactions; both types of processes require lateral mobility and, quite often, mutual reorganization of membrane-associated components.

Supported lipid bilayers can be created by the self assembly of lipids into bilayers on solid supports, typically glass. This planar configuration is ideally suited for observation by fluorescence microscopy. A key finding is that the lipid molecules in supported membranes retain the lateral fluidity associated with lipid membranes in vesicles and in living cells. Furthermore, living cells recognize components displayed on the surface of supported membranes; thus, if the appropriate components are present, the supported membrane mimics a real cell membrane. It has been shown both by neutron scattering and by NMR on glass beads clad with supported membranes that the bilayer is separated from the solid support by a thin layer of water approximately 10-15 Å thick. This water provides a lubricating layer that maintains the lateral fluidity of both leaflets of the membrane. A poorly understood combination of hydration, van der Waals and electrostatic forces traps the bilayer at the surface, and it is indefinitely stable so long as the entire system is hydrated.

Supported membranes are most often prepared by vesicle fusion onto a substrate. It has been shown that small unilamellar vesicles (typically 25-100 nm diameter) initially adsorb to the surface; at low surface coverage, these adsorbed vesicles are stable on the surface. At high coverage, the vesicles rupture and fuse to form the supported membrane. An intermediate in this process has been visualized by atomic force microscopy (AFM) on mica surfaces. These measurements suggest that when small vesicles are close enough they fuse to form larger vesicles and that these rupture and ultimately fuse.

When cell recognition components are incorporated in the supported membrane, cell-surface interactions and their functional consequences can be studied. Such uses complement surface patterning with ligands that direct cell growth and/or stimulate function. These bioactive ligands are attached covalently to the surface and consequently are laterally immobile. Ligands of the type used in some of these experiments have recently been attached to lipid-like molecules and incorporated into supported membranes. These surfaces were then used to study factors that control cell adherence and spreading. Hybrid surfaces have also been created in which some regions are fixed and others are mobile.

If functional membrane proteins such as ion channels and hormone receptors could be displayed in arrays, this would be of widespread interest in the pharmaceutical industry for high-throughput screening of membrane-associated drug targets, a huge area of interest. It may be possible to integrate patterning, electrophoresis and electronic detection methods on a single surface. Using fabrication methods for controlling membrane assembly and composition, supported membranes may also be used as a template for the assembly of more complex structures combining synthetic and living components. Other practical applications are the design of highly selective receptor surfaces of biosensors on electrooptical devices or the biofunctionalization of inorganic solids.

Supported lipid bilayers provide a convenient format for such studies that captures lateral fluidity and other properties of biological membranes. Such systems currently provide a robust tool for the study of a variety of systems including integrins, gap junctions, ion channels, GPI-anchored proteins, synthetic peptides, and cells of the immune system.

However, interpretation of experimental results is complicated by the presence of target biomolecules that are not mobile within the bilayer plane and thus not representative of their mobile, cellular counterparts. The problem is addressed by the present invention.

### RELATED PUBLICATIONS

U.S. Pat. No. 6,228,326, Boxer et al. Supported membranes are reviewed by Sackmann (1996) Science 271(5245):43-8. The use of supported planar membranes in studies of cell-cell recognition are discussed by McConnell et al. (1986) Biochimica et Biophysica Acta 864(1): 95-106. Ligand accessibility as means to control cell response to bioactive bilayer membranes is discussed by Dori et al. (2000) Journal of Biomedical Materials Research 50(1): 75-81. Bayerl and Bloom (1990) Biophysical Journal 58(2): 357-362describe physical-properties of single phospholipid-bilayers adsorbed to micro glass-beads. Johnson et al. (1991), Structure of an Adsorbed Dimyristoylphosphatidylcholine Bilayer Measured With Specular Reflection of Neutrons. Biophysical Journal, 1991. 59(2): p. 289-294.

Koenig et al. (1996) Langmuir 12(5):1343-1350 describe neutron reflectivity and atomic force microscopy studies of a lipid bilayer in water adsorbed to the surface of a silicon single crystal. Tethered polymer-supported planar lipid bilayers for reconstitution of integral membrane proteins are discussed by Wagner and Tamm (2000) Biophysical Journal 79(3): 1400-1414.

Patterning, micropatterning and electrical manipulation of fluid lipid blayers is described by Groves et aL (1996) Biophys. J. 71 2716-2723; Groves et al. (1997) Science, 275:651-653; Hovis and Boxer (2000) Langmuir 16(3): 894-897; Kung et al. (2000) Langmuir 16(17): 6773-6776; Hovis and Boxer (2001) Langmuir 17(11): 3400-3405; and Kam and Boxer (2000) Journal of the American Chemical Society 122(51): 12901-12902.

Localization and separation of biomolecules is discussed by van Oudenaarden and Boxer (1999) Science 285:1046-1048; Chao et al. (1981) Biophysical Journal 36(1): 139-53; Lin-Liu et al. (1984) Biophysical Journal 45(6):1211-7; Cremer et al. (1999) Langmuir 15:3893-3896; Groves et al. (1997) Proc. Natl. Acad. Sci. 94: 13390-13395; Kam and Boxer (2001) Journal of Biomedical Materials Research 55(4): 487-495; and Groves and Boxer (1995) Biophys. J. 69:1972-1975.

### SUMMARY OF THE INVENTION

The invention described here is a general and robust method for generating mobile, membrane-associated biomolecules in a supported membrane, by the process of sequestering and collecting mobile and immobile populations of biomolecules. Two connected patches of supported lipid bilayers may contain target biomolecules, which include both mobile and immobile molecules. Populations of the target biomolecule that are mobile, but not those that are immobile, can be moved to a region through a variety of mechanisms, including passive diffusion or induced drift, generating a pure, mobile population of biomolecules. In some embodiments of the invention, complex substrates are utilized, e.g. substrates containing switchable barriers to lipid diffusion, including fluidic channels; removal of regions of lipid bilayer by blotting, and the like.

### BRIEF DESCRIPTION OF THE DRAWINGS

FIGS. 1A, 1B and 1C are schematics illustrating the purification of mobile proteins.

FIGS. 2A, 2B and 2C: Implementation of lateral structuring using fluidic channels. (2A) A converging flow system was used to generate two connected regions of lipid bilayer of different composition. Vesicles introduced through one of the channels (on the left in panel A) contained hEFG, a GPI-tethered protein (see Materials and Methods), indicated by the “y”-shaped forms in subsequent panels. Vesicles introduced through the right channel were composed of Egg PC with a small amount of fluorescently labeled lipid, which are indicated by the dark or red headgroups and used for visualization only. (2B) These two types of vesicles impinged on opposite halves of a micropatterned corral, in this case measuring 400 μm×1 mm. (2C) These vesicles rupture on the corral surface, producing the final configuration; the local composition of the bilayer is dictated by the distribution of vesicles.

FIGS. 3A-3I: Electrophoresis and purification of mobile proteins. (3A) A micrograph illustrating a 400 μm×1 mm corral produced using the strategy illustrated in FIGS. 3A-C. On the left side of the corral, the bilayer was formed only from vesicles containing hEFG (green). The right side of the corral was formed from vesicles of Egg PC (containing a small amount of NBD-labeled lipid, shown in red). For clarity in the next panels, the red NBD signal is omitted. (3B, 3C) Under the influence of a 20 V/cm electric field applied across the sample for 3 hr., a population of the hEFG molecules migrated to the right-hand side of the corral, and accumulated against the right-hand wall. On the right hand side of the corral, a photobleached spot of hEFG (3D) dissipated over a time course of 10 minutes (3E), demonstrating that these molecules comprise a mobile population of hEFG. The line profiles shown in 3F supports this observation. At the beginning of this time period, the electric field was turned off to avoid migration of the spot. In addition to diffusion of the photobleach spot over this time period, hEFG that accumulated against the right side wall also diffused, further demonstrating mobility of these molecules. By contrast, a 70-μm photobleach spot on the left side of the corral (3G) did not dissipate over a 10-minute period, as demonstrated by the image in panel H and the profiles shown in 31, indicating that the hEFG proteins retained on the left side of the corral remain immobile. The intensity profiles presented in 3F and 31 were taken vertically across the 70-μm photobleach spot.

### DETAILED DESCRIPTION OF THE EMBODIMENTS

The invention described here is a general and robust method for generating mobile, membrane-associated biomolecules in a supported membrane, by the process of sequestering and collecting mobile and immobile populations of biomolecules. Two connected patches of supported lipid bilayers may contain target biomolecules, which include both mobile and immobile molecules. Populations of the target biomolecule that are mobile, but not those that are immobile, can be moved to a region through a variety of mechanisms, including passive diffusion or induced drift, generating a pure, mobile population of biomolecules. In some embodiments of the invention, complex substrates are utilized, e.g. substrates containing switchable barriers to lipid diffusion, including fluidic channels; removal of regions of lipid bilayer by blotting, and the like.

Such lateral structuring of mobile membrane-associated biomolecules can be achieved using fluidic channels, where a converging flow system generates connected regions of lipid bilayers of different composition. Vesicles introduced through a channel will impinge on a micropatterned structure, e.g. a corral, and will rupture on the corral surface. Under the influence of an electric field applied across the sample, mobile components are induced to migrate, and accumulate at a wall of the micropatterned structure. Vesicles also fuse where the interface of the two converging flows meets the corral surface, resulting in a transitional, connecting region of bilayer between the two halves. In this embodiment, the transitional region is narrow compared to the corral width. The criterion of transition width versus corral size differentiates from other uses of fluidic channels in creating laterally structured supported bilayer systems.

Purity in this context refers not to chemical composition, but to a population of biomolecules that all exhibit the physical property of mobility in the membrane plane. These methods take advantage of techniques to create laterally structured lipid bilayers and are readily adaptable to a variety of contemporary systems for examining membrane-associated molecules. Since this approach is based on selecting the biomolecules that are mobile and not addressing the specific factors that lead to protein immobility, which may be specific to the target biomolecule, the methods described in this invention are very robust and may be particularly suited for high-throughput screening of membrane proteins. Lastly, the concepts demonstrated here are directly adaptable to systems other than glass-supported lipid bilayers, such as tethered bilayers and supported monolayers.

Membrane-tethered biomolecules. One of the most successful and general strategies for integrating target biomolecules into supported lipid bilayers is to first incorporate the biomolecule into lipid vesicles which then fuse together on an appropriate substrate to produce the supported membrane. While this strategy can yield a population of biomolecules that are mobile within the supported bilayer, a significant fraction, typically 10-40% are immobile, especially when these biomolecules are membrane-associated proteins. The factors influencing the mobility of biomolecules in supported bilayers are not well understood and, to date, no general strategy for completely eliminating the immobile fraction has been described. One approach to enhance mobility is to tether or cushion the supported bilayer, which increases the distance between the hard support and the fluid bilayer. This strategy can increase the fraction of membrane-associated proteins that exhibits lateral mobility; however, a substantial immobile fraction remains.

Laterally structured lipid bilayers. The development of laterally structured lipid bilayers has added new functionality and utility to supported membrane systems. For example, tools have been developed for micropatterning supported bilayers, including patterning of material barriers and stamping/blotting of lipid bilayers, in order to control lateral diffusion of lipids. These studies have yielded a range of novel structures, such as the two-dimensional Brownian ratchet, and lipid bilayer arrays containing multiple, separate patches of lipid bilayer on a single surface. Of particular relevance to the invention described here, microfluidics concepts are used to control the distribution of vesicles in solution and, consequentially, the lateral composition of supported lipid bilayers. The first systems that were created using these techniques are gradient arrays in which different regions of the same surface are of different composition.

Under the influence of an applied electric field, charged biomolecules associated with cellular membranes can be induced to move along the bilayer plane, accumulating on one side of the cell. In a similar fashion, charged biomolecules associated with supported lipid bilayers can be moved under the influence of an applied electric field; for example this form of electrophoresis can be used as a driving force for either inducing biomolecular motion through a Brownian ratchet or accumulation of biomolecules against diffusion barriers in the context of concentrating and/or separating membrane components.

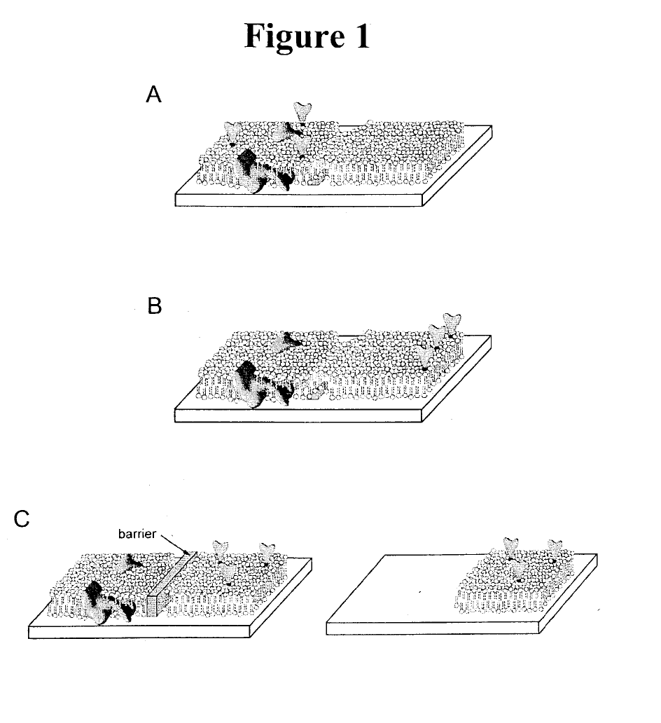

Biomolecules on laterally heterogenous surfaces. An important property of fluid supported lipid bilayers is that mobile biomolecules in one patch of membrane can diffuse into any connected region. In the invention described here, we use this property in conjunction with lateral structuring to separate mobile target biomolecules from those that are immobile. Again, membrane fluidity is critical to the functioning of a large number of membrane proteins, and the immobility of such a protein reconstituted into lipid bilayers suggests that this protein is not representative of the idealized target; the generation of pure populations of mobile proteins, which may have the same chemical composition as the immobile biomolecules but exhibit different physical behavior (i.e., mobility), is a valuable process. The most basic configuration consists of two connected regions of lipid bilayer of different composition, as illustrated in FIG. 1A. Several of the techniques for manipulating lipid bilayers, including membrane stamping and (as will be demonstrated later in this disclosure) fluidic patterning, provide mechanisms for creating this basic configuration.

One of these regions, region 1 on the left side of FIG. 1A, contains both mobile and immobile populations of a target biomolecule, which are illustrated schematically by the upright “y”-shaped and membrane-embedded shapes, respectively. While trapping of a biomolecule to glass may be one mechanism by which proteins become immobile on solid supports, these drawings are not intended to explain or identify all of the factors that may influence protein mobility, and the concepts described here are not limited to any one specific mechanism.

The other region, region 2 on the right side of FIG. 1A, consists of lipid alone. Members of the mobile population of target biomolecules, but not of the immobile population, can move from region 1 to region 2; this process can be through either passive diffusion or induced drift (by application of an electric field, for example) as illustrated in FIG. 1B. After this process, region 2 contains a purified population of mobile target biomolecules that can be subsequently used in binding assays, collected, or studied for other properties. Immobile biomolecules remain in region 1, and can be either discarded or studied further.

Two alternative embodiments are illustrated in FIG. 1C. First (on the left of FIG. 1C), more complex surfaces containing such structures are switchable barriers allow sequestering and concentration of mobile biomolecules to specific regions of lipid bilayer. Second, removal of lipid bilayer containing immobile biomolecules can be accomplished by blotting, completely removing the immobile protein from the surface. This method allows introduction of a second type of bilayer onto the newly cleaned surface or other novel systems.

Lipid vesicles and lipid bilayers. The lipid bilayer, which may be referred to interchangeably as a membrane, is an essential structure in biology. It is an ordered structure of two opposing layers of lipids, with the polar “head” groups located on the surface of an aqueous medium and the hydrophobic tails aligned in the internal space. Each layer of the membrane may be referred to as a leaflet. Spherical vesicles, or liposomes, form spontaneously by dispersing phospholipids in an aqueous medium. These spherical structures have a diameter of up to about 1 μm, and can enclose concentric lipid bilayers, and the aqueous medium. An important property of lipid bilayers is that they spontaneously tend to seal to form closed structures.

The terms “immobilized” or “supported” are used herein, for purposes of the specification and claims, to mean adsorption, coating or bonding the biological membrane according to the present invention to a support surface or structure.

As will be discussed in more detail, the lipid bilayer, either when in the form of a vesicle or supported on a capillary, can also comprise non-lipid components, e.g. proteins, fluorescent compounds, compounds for screening as targets, etc., usually in binding assays, where the non-lipid component is a member of a specific binding pair. Such compounds can be introduced during the initial formation of vesicles, or can readily be added to a capillary supported bilayer. For example, proteins can be attached by ionic bonds or calcium bridges to the electrically charged phosphoryl surface of the bilayer, or bound within the phospholipid bilayer, and may extend through and bind to the fatty acid internal regions of the membrane. Membranes containing such proteins and other compounds can be provided by simply forming the initial vesicles in the presence of such proteins or other compounds.

Bilayer-forming lipids. There are a variety of synthetic and naturally-occurring bilayer or vesicle-forming lipids, including the phospholipids, such as phosphatidylcholine (PC), phosphatidylethanolamine (PE), phosphatidylserine(PS), phosphatidicacid, phosphatidylinositol (PI), phosphatidylglycerol (PG), and sphingomyelin, where the two hydrocarbon chains are typically between about 14-22 carbon atoms in length, and have varying degrees of unsaturation. Other suitable lipids include glycolipids and sterols such as cholesterol. Diacyl-chain lipids suitable for use in the present invention include diacyl glycerol, phosphatidyl ethanolamine (PE) and phosphatidylglycerol (PG). These lipids and phospholipids can be obtained commercially or prepared according to published methods.

The aqueous film medium used in the formation of vesicles, and the aqueous layer that supports the bilayer, may be any suitable aqueous solution, such as a buffered saline solution (e.g., PBS). The medium can be readily changed (taking care, of course, to keep the supported bilayer submerged at all times) by, e.g., flow-through rinsing.

Lipids for use in a supported membrane can be readily modified by a variety of methods known in the art. For example, polymer-lipid conjugates can be diffused into preformed liposomes or supported membranes, e.g. by adding a solution containing a concentrated dispersion of micelles of polymer-lipid conjugates to the vesicles or lipid bilayers, and incubating under conditions effective to achieve insertion of the modified lipids. Alternatively, a conjugate can be formed with suitable lipids prior to initial formation of the liposome vesicles. In another method, a vesicle-forming lipid activated for covalent attachment of a biomolecule is incorporated into the membrane, which is then conjugated to the desired compound.

A variety of methods are available for preparing a conjugate composed of a molecule of interest and a vesicle-forming lipid. For example, water-soluble, amine-containing compounds can be covalently attached to lipids, such as phosphatidylethanolamine, by reacting the amine-containing compound with a lipid that has been derivatized to contain an activated ester of N-hydroxysuccinimide.

Another method involves Schiff-base formation between an aldehyde group on a lipid, typically a phospholipid, and a primary amino acid on the compound, for example a protein. The aldehyde group is preferably formed by periodate oxidation of the lipid. The coupling reaction, after removal of the oxidant, is carried out in the presence of a reducing agent, such as dithiotreitol. Typical aldehyde-lipid precursors suitable in the method include lactosylceramide, trihexosylceramide, galactocerebroside, phosphatidylglycerol, phosphatidylinositol and gangliosides.

A second general coupling method is applicable to thiol-containing compounds, and involves formation of a disulfide or thioether bond between a lipid and the compound. In the disulfide reaction, a lipid amine, such as phosphatidyl-ethanolamine, is modified to contain a pyridyiditho derivative which can react with an exposed thiol group in the biomolecule. The thioether coupling method, described by Martin (1982), is carried out by forming a sulfhydryl-reactive phospholipid, such N-(4)P-maleimido-phenyl(butyryl)phosphatidylethanolamine, and reacting the lipid with the thiol-containing compound. Another method for reacting a biomolecule with a lipid involves reacting the biomolecule with a lipid which has been derivatized to contain an activated ester of N-hydroxysuccinimide. The reaction is typically carried out in the presence of a mild detergent, such as deoxycholate. Like the reactions described above, this coupling reaction is preferably performed prior to incorporating the lipid into the liposome.

Methods for attachment of a compound to a lipid through a short spacer arm have been described, such as in U.S. Pat. No. 4,762,915. In general, attachment of a moiety to a spacer arm can be accomplished by derivatizing the vesicle-forming lipid, typically distearol phosphatidylethanolamine (DSPE), with a hydrophilic polymer, such as polyethylene glycol (PEG), having a reactive terminal group for attachment of an affinity moiety. Methods for attachment of ligands to activated PEG chains are described in the art. In these methods, the inert terminal methoxy group of mPEG is replaced with a reactive functionality suitable for conjugation reactions, such as an amino or hydrazide group. The end functionalized PEG is attached to a lipid, typically DSPE. The functionalized PEG-DSPE derivatives are employed in liposome formation and the desired ligand (i.e., biomolecule) is attached to the reactive end of the PEG chain before or after liposome formation.

Another method of linking compounds, e.g. proteins, peptides, etc. to a supported lipid bilayer is via specific interactions between the side chain of the amino acid histidine and divalent transition metal ions immobilized on the membrane surface. This method has been used, for example, to attach various proteins and peptides to lipid monolayers. A genetic sequence encoding the polypeptide of interest is modified to insert a poly-histidine (e.g., hexa-histidine) tag at one of its termini (e.g., the C-terminus). The lipid bilayer is formed of, or derivatized with, metal-chelating moieties, e. g. copper-chelating moieties or lipids, and the expressed His-tagged polypeptide is incubated with the vesicles used to generate the supported bilayer, or with the supported bilayer itself.

Specific high-affinity molecular interactions may also be employed to link selected compounds to a supported bilayer. For example, a bilayer expanse may be formed to include biotinylated lipids (available from, e.g., Molecular Probes, Eugene, OR), and a compound linked or coupled to avidin or steptavidin may be linked to the bilayer via the biotin moieties.

Compounds may also be linked to a supported lipid bilayer via glycan-phosphatidyl inositol (GPI). Polypeptides to be linked can be genetically engineered to contain a GPI linkage (Caras, et al., 1987; Whitehorn, et al., 199S). Incorporation of a GPI attachment signal into a coding sequence will cause the encoded polypeptide to be post-translationally modified by the cell resulting in a GPI linkage at the signal position. It will be appreciated that this type of alteration generally does not affect the molecular recognition properties of proteins.

Specific Binding pair. The term “specific binding member” as used herein refers to a member of a specific binding pair, i.e. two molecules, usually two different molecules, where one of the molecules through chemical or physical means specifically binds to the other molecule, and where the binding of the members of the specific binding pair is at a substantially higher affinity than random complex formation. Generally, the binding affinity will be at least about Kₘ>10⁵. The complementary members of a specific binding pair are sometimes referred to as a ligand and receptor. Such binding pairs may include antigen and antibody specific binding pairs, peptide-MHC antigen and T cell receptor pairs; biotin and avidin or streptavidin; carbohydrates and lectins; complementary nucleotide sequences (including nucleic acid sequences used as probes and capture agents in DNA hybridization assays); peptide ligands and receptor; effector and receptor molecules; hormones and hormone binding protein; enzyme cofactors and enzymes; enzyme inhibitors and enzymes; and the like. The specific binding pairs may include analogs, derivatives and fragments of the original specific binding member. Biological receptors are often associated with lipid bilayer membranes, such as the extracellular, golgi or nuclear membranes. Receptors for incorporation into lipid bilayers of the invention can be isolated from natural sources, recombinantly expressed, synthesized in vitro, etc.

Binding is generally considered to be specific if it results from a molecular interaction between two binding sites, rather than from “non-specific” stickiness of the molecules. Specificity of reversible binding can be confirmed by competing off labeled ligand with an excess of unlabeled ligand according to known methods. Non-specific interactions can be minimized by including an excess of a reagent, e.g. BSA, that does not have binding sites for either the ligand or receptor.

Compounds of interest for use as the lipid-bound or mobile binding member include biologically active agents of numerous chemical classes, primarily organic molecules, which may include organometallic molecules, inorganic molecules, genetic sequences, etc. Candidate agents comprise functional groups necessary for structural interaction with proteins, particularly hydrogen bonding, and typically include at least an amine, carbonyl, hydroxyl or carboxyl group, frequently at least two of the functional chemical groups. The candidate agents often comprise cyclical carbon or heterocyclic structures and/or aromatic or polyaromatic structures substituted with one or more of the above functional groups. Candidate agents are also found among biomolecules, including peptides, polynucleotides, saccharides, fatty acids, steroids, purines, pyrimidines, derivatives, structural analogs or combinations thereof.

Included are pharmacologically active drugs, genetically active molecules, etc. Compounds of interest include chemotherapeutic agents, anti-inflammatory agents, hormones or hormone antagonists, ion channel modifiers, and neuroactive agents. Exemplary of pharmaceutical agents suitable for this invention are those described in, “The Pharmacological Basis of Therapeutics,” Goodman and Gilman, McGraw-Hill, New York, N.Y., (1996), Ninth edition, under the sections: Drugs Acting at Synaptic and Neuroeffector Junctional Sites; Drugs Acting on the Central Nervous System; Autacoids: Drug Therapy of Inflammation; Water, Salts and Ions; Drugs Affecting Renal Function and Electrolyte Metabolism; Cardiovascular Drugs; Drugs Affecting Gastrointestinal Function; Drugs Affecting Uterine Motility; Chemotherapy of Parasitic Infections; Chemotherapy of Microbial Diseases; Chemotherapy of Neoplastic Diseases; Drugs Used for Immunosuppression; Drugs Acting on Blood-Forming organs; Hormones and Hormone Antagonists; Vitamins, Dermatology; and Toxicology, all incorporated herein by reference. Also included are toxins, and biological and chemical warfare agents, for example see Somani, S. M. (Ed.), “Chemical Warfare Agents,” Academic Press, New York, 1992).

The term samples also includes the fluids described above to which additional components have been added, for example components that affect the ionic strength, pH, total protein concentration, etc. In addition, the samples may be treated to achieve at least partial fractionation or concentration. Biological samples may be stored if care is taken to reduce degradation of the compound, e.g. under nitrogen, frozen, or a combination thereof. The volume of sample used is sufficient to allow for measurable detection, usually from about 0.1:l to 1 ml of a biological sample is sufficient.

Compounds are obtained from a wide variety of sources including libraries of synthetic or natural compounds. For example, numerous means are available for random and directed synthesis of a wide variety of organic compounds, including biomolecules, including expression of randomized oligonucleotides and oligopeptides. Alternatively, libraries of natural compounds in the form of bacterial, fungal, plant and animal extracts are available or readily produced. Additionally, natural or synthetically produced libraries and compounds are readily modified through conventional chemical, physical and biochemical means, and may be used to produce combinatorial libraries. Known pharmacological agents may be subjected to directed or random chemical modifications, such as acylation, alkylation, esterification, amidification, etc. to produce structural analogs.

Labels and Detection. Detection of specific binding may utilize a wide variety of techniques, as known in the art. For example, binding can result in a detectable change in the conformation of one or both of the binding pair members, e.g. the opening of an ion channel associated with or part of the receptor; or may result in a change in the immediate environment of the member e.g., detection of binding by surface plasmon resonance. Alternatively one of the binding pair members may comprise a detectable label. Directly detectable labels include isotopic labels, in which one or more of the nucleotides is labeled with a radioactive label, such as ³²S, ₃₂P, ₃H, etc.

A wide variety of fluorescers may be employed either by themselves or in conjunction with quencher molecules. Fluorescers of interest fall into a variety of categories having certain primary functionalities. These primary functionalities include 1- and 2-aminonaphthalene, p,p′-diaminostilbenes, pyrenes, quaternary phenanthridine salts, 9-aminoacridines, p,p′-diaminobenzophenone imines, anthracenes, oxacarbocyanine, merocyanine, 3-aminoequilenin, perylene, bis-benzoxazole, bis-p-oxazolyl benzene, 1,2-benzophenazin, retinol, bis-3-aminopyridinium salts, hellebrigenin, tetracycline, sterophenol, benzimidzaolylphenylamine, 2-oxo-3-chromen, indole, xanthen, 7-hydroxycoumarin, phenoxazine, salicylate, strophanthidin, porphyrins, triarylmethanes and flavin. Individual fluorescent compounds which have functionalities for linking or which can be modified to incorporate such functionalities include, e.g., dansyl chloride; fluoresceins such as 3,6-dihydroxy-9-phenylxanthhydrol; rhodamineisothiocyanate; N-phenyl 1-amino-8-sulfonatonaphthalene; N-phenyl 2-amino-6-sulfonatonaphthalene; 4-acetamido-4-isothiocyanato-stilbene-2,2′-disulfonic acid; pyrene-3-sulfonic acid; 2-toluidinonaphthalene-6-sulfonate; N-phenyl, N-methyl 2-aminoaphthalene-6-sulfonate; ethidium bromide; stebrine; auromine-0,2-(9′-anthroyl)palmitate; dansyl phosphatidylethanolamine; N,N′-dioctadecyl oxacarbocyanine; N,N′-dihexyl oxacarbocyanine; merocyanine, 4-(3′pyrenyl)butyrate; d-3-aminodesoxy-equilenin; 12-(9′-anthroyl)stearate; 2-methylanthracene; 9-vinylanthracene; 2,2′-(vinylene-p-phenylene)bisbenzoxazole; p-bis[2-(4-methyl-5-phenyl-oxazolyl)]benzene; 6-dimethylamino-1,2-benzophenazin; retinol; bis(3′-aminopyridinium) 1,10-decandiyl diiodide; sulfonaphthylhydrazone of hellibrienin; chlorotetracycline; N-(7-dimethylamino-4-methyl-2-oxo-3-chromenyl)maleimide; N-[p-(2-benzimidazolyl)-phenyl]maleimide; N-(4-fluoranthyl)maleimide; bis(homovanillic acid); resazarin; 4-chloro-7-nitro-2,1,3-benzooxadiazole; merocyanine 540; resorufin; rose bengal; and 2,4-diphenyl-3(2H)-furanone.

Desirably, fluorescers should absorb light above about 300 nm, preferably about 350 nm, and more preferably above about 400 nm, usually emitting at wavelengths greater than about 10 nm higher than the wavelength of the light absorbed. It should be noted that the absorption and emission characteristics of the bound dye may differ from the unbound dye. Therefore, when referring to the various wavelength ranges and characteristics of the dyes, it is intended to indicate the dyes as employed and not the dye which is unconjugated and characterized in an arbitrary solvent.

Detectable signal may also be provided by chemiluminescent and bioluminescent sources. Chemiluminescent sources include a compound which becomes electronically excited by a chemical reaction and may then emit light which serves as the detectible signal or donates energy to a fluorescent acceptor. A diverse number of families of compounds have been found to provide chemiluminescence under a variety of conditions. One family of compounds is 2,3-dihydro-1,-4-phthalazinedione. The most popular compound is luminol, which is the 5-amino compound. Other members of the family include the 5-amino-6,7,8-trimethoxy- and the dimethylamino[ca]benz analog. These compounds can be made to luminesce with alkaline hydrogen peroxide or calcium hypochlorite and base. Another family of compounds is the 2,4,5-triphenylimidazoles, with lophine as the common name for the parent product. Chemiluminescent analogs include para-dimethylamino and -methoxy substituents. Chemiluminescence may also be obtained with oxalates, usually oxalyl active esters, e.g., p-nitrophenyl and a peroxide, e.g., hydrogen peroxide, under basic conditions. Alternatively, luciferins may be used in conjunction with luciferase or lucigenins to provide bioluminescence.

Spin labels are provided by reporter molecules with an unpaired electron spin which can be detected by electron spin resonance (ESR) spectroscopy. Exemplary spin labels include organic free radicals, transitional metal complexes, particularly vanadium, copper, iron, and manganese, and the like. Exemplary spin labels include nitroxide free radicals.

Devices. For the purposes of this invention, a device may comprise a supported lipid bilayer comprising pure populations of mobile membrane-associated biomolecules, e.g. in an information processing system, and may comprise multiple supported membranes, e.g. an array. The lipid bilayer is supported on a surface, allowing for one or more binding partners of the membrane to interact by specific binding with a mobile reactant. Upon the molecular interaction with the reactant and binding partner, a signal is sent, thereby allowing for qualitative and quantitative characterization of such molecular interaction.

In devices employing electrical detection, the support grid preferably contains a conductive electrode and electronic lead for each array element of the device. The leads typically terminate as extensions or “pins” from the device, which can be interfaced with a connector cable or ribbon leading to a processor. The electrodes preferably form at least a portion of the bilayer-compatible surface and are separated from one another by strips of insulating material. They can be used to detect capacitative as well as conductive current transients. In one embodiment, the electrodes form a portion of the bilayer-compatible surface. In another embodiment the electrodes are positioned just beneath the bilayer-compatible surface, i. e., the electrode surface is coated with a thin layer of material, such as low-temperature grown oxide (e.g., SiO₂), which forms the bilayer-compatible surface. In embodiments where this layer is an insulating material, it is preferably less than about 1 μm in thickness to enable the detection of capacitative transients cased by binding of ligands to ionophoric receptors.

Microfluidic devices or systems may include at least one such supported lipid bilayer, and further comprises integrated microfluidic channels for the flow of fluids and reactants within the device. Generally such a device has an integrated format, i.e. the body structure of the device comprises an aggregation of separate parts, e.g., capillaries, joints, chambers, layers, etc., which are appropriately mated or joined together. Typically, the devices will comprise a top portion, a bottom portion, and an interior portion, wherein the interior portion substantially defines the channels and chambers of the device. Generally, the bottom portion will comprise a solid substrate that is substantially planar in structure, and which has at least one substantially flat upper surface. A variety of substrate materials may be employed as the bottom portion.

Typically, because the devices are microfabricated, substrate materials will generally be selected based upon their compatibility with known microfabrication techniques, e.g., photolithography, wet chemical etching, laser ablation, air abrasion techniques, injection molding, embossing, and other techniques. The substrate materials are also generally selected for their compatibility with the full range of conditions to which the microfluidic devices may be exposed, including extremes of pH, temperature, salt concentration, and application of electric fields. Accordingly, in some preferred aspects, the substrate material may include materials normally associated with the semiconductor industry in which such microfabrication techniques are regularly employed, including, e.g., silica based substrates such as glass, quartz, silicon or polysilicon, as well as other substrate materials, such as gallium arsenide and the like. In the case of semiconductive materials, it will often be desirable to provide an insulating coating or layer, e.g., silicon oxide, over the substrate material, particularly where electric fields are to be applied.

The substrate materials may comprise polymeric materials, e.g., plastics, such as polymethylmethacrylate (PMMA), polycarbonate, polytetrafluoroethylene (TEFLON.TM.), polyvinylchloride (PVC), polydimethylsiloxane (PDMS), polysulfone, and the like. Such substrates are readily manufactured from microfabricated masters, using well known molding techniques, such as injection molding, embossing or stamping, or by polymerizing the polymeric precursor material within the mold. Such polymeric substrate materials are preferred for their ease of manufacture, low cost and disposability, as well as their general inertness to most extreme reaction conditions. Again, these polymeric materials may include treated surfaces, e.g., derivatized or coated surfaces, to enhance their utility in the microfluidic system.

The channels and/or chambers of the microfluidic devices are typically fabricated into the upper surface of the substrate, or bottom portion, using the above described microfabrication techniques, as microscale grooves or indentations. The lower surface of the top portion of the microfluidic device, which top portion typically comprises a second planar substrate, is then overlaid upon and bonded to the surface of the bottom substrate, sealing the channels and/or chambers (the interior portion) of the device at the interface of these two components. Bonding of the top portion to the bottom portion may be carried out using a variety of known methods, depending upon the nature of the substrate material. For example, in the case of glass substrates, thermal bonding techniques may be used which employ elevated temperatures and pressure to bond the top portion of the device to the bottom portion. Polymeric substrates may be bonded using similar techniques, except that the temperatures used are generally lower to prevent excessive melting of the substrate material. Alternative methods may also be used to bond polymeric parts of the device together, including acoustic welding techniques, or the use of adhesives, e.g., UV curable adhesives, and the like.

In preferred aspects, the devices, methods and systems described herein, employ electrokinetic material transport systems, and preferably, controlled electrokinetic material transport systems. As used herein, “electrokinetic material transport systems” include systems which transport and direct materials within an interconnected channel and/or chamber containing structure, through the application of electrical fields to the materials, thereby causing material movement through and among the channel and/or chambers, i.e., cations will move toward the negative electrode, while anions will move toward the positive electrode.

Such electrokinetic material transport and direction systems include those systems that rely upon the electrophoretic mobility of charged species within the electric field applied to the structure. Other electrokinetic material direction and transport systems rely upon the electroosmotic flow of fluid and material within a channel or chamber structure which results from the application of an electric field across such structures.

The device is optionally connected to or interfaced with a processor, which stores and/or analyzes signals from binding events. The processor in turn forwards the data to computer memory (either hard disk or RAM) from where it can be used by a software program to further analyze, print and/or display the results.

A device such as described above can be used to detect low concentrations of biologically-active analytes or ligands in a solution containing a complex mixture of ligands. In such a method, the device is constructed with different receptors in the different bilayers. To control for signal fluctuations, several different array elements may contain the same type of receptor. Similarly, designated array elements may be used for positive and/or negative control purposes. The array device is then contacted with an aqueous solution containing a mixture of ligands to be analyzed for the presence of selected ligands, where the mixture is flowed through the array. When a selected ligand specifically binds to a receptor, the binding is detected by a suitable detection method.

Arrays of the subject supported lipid bilayers can be used as substrates for holding an array of binding members employed in screens of compounds. In particular, high-throughput screens of large libraries of compounds are typically optimized for speed and efficiency in order to rapidly identify candidate compounds for bioactivity testing. Devices of the invention may be used to assess the bioactivity of compounds identified in a high-throughput screen. In devices employing electrical detection using electrodes in each of the array elements, it will be appreciated that since a water film separates the electrode from the bilayer, an electric field may be applied across the bilayer membrane, e.g., to activate voltage-dependent ion channels. This allows screening for compounds which only bind to the channel when the channel is in a state other than the resting state (e.g., in an activated or inactivated state).

In a related embodiment, devices of the invention are used as substrates for holding libraries (e.g., combinatorial libraries) of compounds.

It is to be understood that this invention is not limited to the particular methodology, protocols, device, and reagents described, as such may vary. It is also to be understood that the terminology used herein is for the purpose of describing particular embodiments only, and is not intended to limit the scope of the present invention which will be limited only by the appended claims.

As used herein the singular forms “a”, “and”, and “the” include plural referents unless the context clearly dictates otherwise. Thus, for example, reference to “a capillary” includes a plurality of such capillaries and reference to “the biosensor” includes reference to one or more biosensors and equivalents thereof known to those skilled in the art, and so forth. All technical and scientific terms used herein have the same meaning as commonly understood to one of ordinary skill in the art to which this invention belongs unless clearly indicated otherwise.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are now described. All publications mentioned herein are herein incorporated by reference to disclose and describe the methods and/or materials in connection with which the publications are cited.

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Centigrade, and pressure is at or near atmospheric.

While the present invention has been described with reference to the specific embodiments thereof, it should be understood by those skilled in the art that various changes may be made and equivalents may be substituted without departing from the true spirit and scope of the invention. In addition, many modifications may be made to adapt a particular situation, material, composition of matter, process, process step or steps, to the objective, spirit and scope of the present invention. All such modifications are intended to be within the scope of the claims appended hereto.

### EXAMPLES

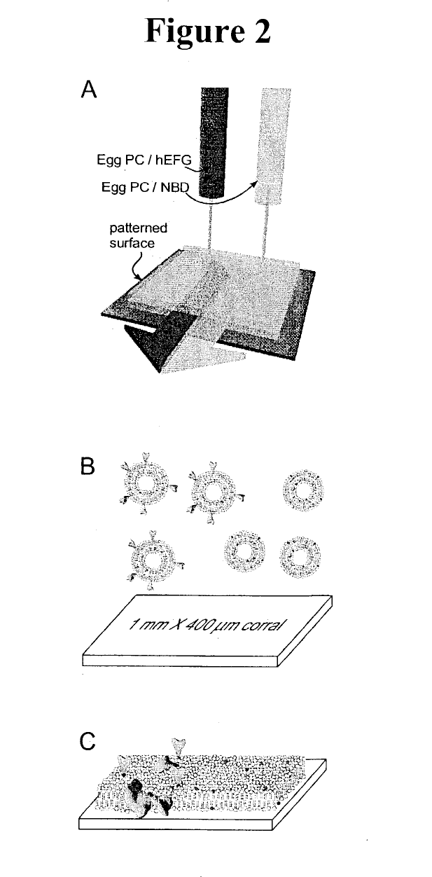

Demonstrated herein is the purification of biomolecules on laterally heterogeneous lipid bilayer surfaces with hEFG, a protein containing a covalently attached lipid moiety (a GPI tether, see Materials and Methods) that facilitates anchorage of this protein to lipid bilayers. A converging flow configuration, illustrated in FIG. 2A, is used to create two connected regions of lipid bilayer, one from vesicles of Egg PC containing hEFG (labeled with a fluorescent dye) and the other from vesicles of Egg PC supplemented with a small amount of fluorescently labeled lipid (included for visualization purposes only). The flows impinge on a surface containing barriers of fibronectin that define a series of rectangular, 400 ∝m×1 mm regions that become “corrals” for assembly of lipid bilayers.

One such corral is represented schematically by the empty substrate in FIG. 2B. This corral is positioned on the surface such that the interface between the two converging flows passes along the short axis of the corral. As illustrated in FIG. 2B, the left half of the corral is thus exposed to vesicles that contain the hEFG protein (indicated by the “y”-shaped forms), while the right half is exposed to hEFG-free vesicles of Egg PC (containing a small amount of labeled lipids, indicated with the dark or red head groups). Each half (left and right) of the resultant bilayer reflects the composition of impinging vesicles.

Vesicles also fuse where the interface of the two converging flows meets the corral surface, resulting in a transitional, connecting region of bilayer between the two halves (FIG. 2C), thus implementing the configuration indicated in FIG. 1A. It is important to note that in this implementation, the transitional region is narrow compared to the corral width. In the original use of this converging flow mechanism, the transitional region extended across many, smaller corrals (i.e., 25-50 ∝m in width); the corrals were used to locally limit biomolecular diffusion, leading to an array of corrals of controlled, varying composition. The criterion of transition width versus corral size differentiates these two uses of fluidic channels in creating laterally structured supported bilayer systems.

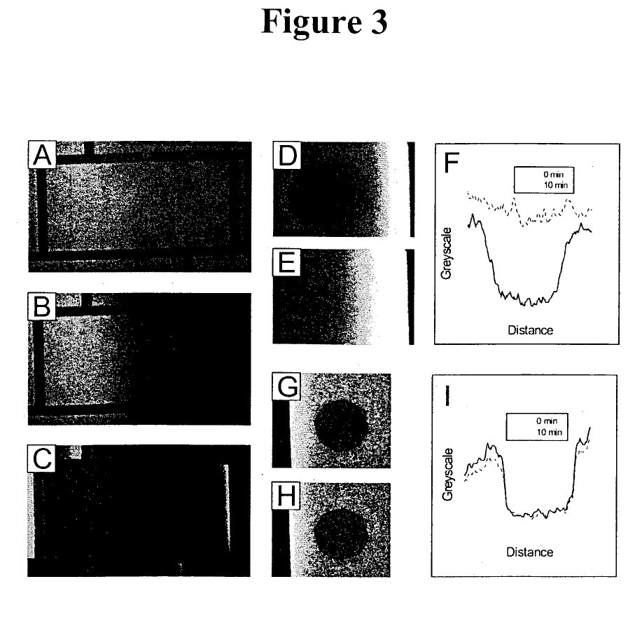

A corral prepared using this procedure is shown in FIG. 3A; the left side of the corral consists of bilayer containing both mobile and immobile molecules of hEFG (in green) while the right side consists of Egg PC (with a small amount of NBD-labeled lipids, shown in red). For clarity of presentation, FIG. 3B shows the same corral as in FIG. 3A, but only the signal associated with hEFG. Based on fluorescence recovery after photobleaching (FRAP) experiments, about 50% of the hEFG proteins in bilayers formed from Egg PC/ hEFG vesicles are mobile.

An electric field of 20 V/cm was applied tangentially across the bilayer to induce drift of the mobile, negatively charged hEFG proteins to the right side of the corral with accumulation against the right hand barrier as shown in FIG. 3C; as in FIG. 3B, the signal associated with the NBD-labeled lipids is omitted for clarity. FRAP experiments showed that a photobleached spot of hEFG molecules on the right side of the corral recovers over a 10-minute period (FIG. 3D,E), indicating that hEFG molecules driven to this side are mobile; the intensity profile shown in FIG. 3F, which was taken vertically across the photobleached spot in FIG. 3D,E, supports this observation and furthermore indicates that there is no appreciable immobile fraction on this side of the corral. In contrast, a photobleach spot on the left side of the corral does not recover (FIG. 3G-I); the hEFG molecules remaining on this side of the corral are immobile.

In summary, we describe and demonstrate a new strategy for generating pure populations of laterally mobile membrane-associated target biomolecules; again, purity in this context refers not to chemical composition, but to a population of biomolecules that all exhibit the physical property of mobility in the membrane plane. While the simple configuration described here is in itself useful, more complex implementations can be considered (FIG. 1C). Importantly, these methods take advantage of techniques to create laterally structured lipid bilayers and are readily adaptable to a variety of contemporary systems for examining membrane-associated molecules. Since this approach is based on selecting the biomolecules that are mobile and not addressing the specific factors that lead to protein immobility, which may be specific to the target biomolecule, the methods described in this invention are very robust and may be particularly suited for high-throughput screening of membrane proteins. Lastly, the concepts demonstrated here are directly adaptable to systems other than glass-supported lipid bilayers, such as tethered bilayers and supported monolayers.

### Materials and Methods

Lipids and proteins. Stock solutions of small unilamellar vesicles (SUV) were prepared by extrusion using standard techniques. Briefly, egg phosphatidylcholine (egg PC) (Avanti Polar Lipids, Alabaster, Ala., USA) was dried from chloroform in glass round-bottom flasks, then desiccated under vacuum for at least 90 minutes. These lipids were reconstituted in HBS (138 mM NaCl/5.3 mM KCl/10 mM HEPES, pH 8.5) at a concentration of 5 mg/ml, and then extruded through 50-nm pore size polycarbonate membranes using a mini-extrusion unit from Avanti. For visualization of lipid bilayers, vesicles of egg PC were supplemented with 1 mol % of a neutral, NBD-labeled lipid (NBD-PE, 1-palmitoyl-2-[12-[(7-nitro-2-1,3-benzoxadiazol-4-yl)amino]dodecanoyl]-sn-glycero-3-phosphoethanolamine; Avanti). Images of these fluorescently labeled protein and lipids were collected on an inverted microscope using appropriate optical filters.

As a model membrane-tethered protein, we used a GPI-modified construct of E-cadherin, called hEFG. This construct consists of the extracellular domain of human E-cadherin attached to a fragment of human immunoglobulin and a signal sequence that, when properly processed in cells, is replaced by a lipid anchor, the GPI moiety. As demonstrated in earlier studies, the GPI anchor is an effective strategy for tethering biomolecules to lipid bilayers. The hEFG protein was incorporated into lipid vesicles using methods similar to those reported in earlier studies. Briefly, the hEFG protein, at a concentration of 100 ∝g/ml in labeling buffer (138 mM NaCl/5.3 mM KCl/10 mM HEPES, pH 7.7 at room temp) supplemented with 1% n-octyl-®-D-gluocopyranoside (Sigma, St. Louis,.Mo., USA), was labeled with an amine-reactive Cy5 dye (Amersham Biosciences, Piscataway, N.J., USA), then dialysed into vesicle solutions against HBS overnight.

Micropattemed surfaces. Glass coverslips measuring 22 mm×40 mm were immersed into a detergent solution (Linbro 7×detergent, diluted 1:5 in deionized water), rinsed extensively with water, then baked at 450° C. for 4 hours. Barriers of fluorescently labeled fibronectin were prepared by microcontact printing as previously described. Two layers of elastomer were hand-cut as described previously and used to create the converging channel setup illustrated in FIG. 2A. Vesicle solutions of Egg PC/hEFG and Egg PC/NBD were manually pumped through this channel across the fibronectin-patterned surface. The substrate was then rinsed extensively in water and mounted in an electrophoresis chamber for subsequent manipulation and microscopy.

What is claimed is:

1. A supported lipid bilayer comprising a substantially pure population of laterally mobile membrane-associated target biomolecules.
